(19) United States
(12) Patent Application Publication
(10) Pub. No.: US 2026/0279501 A1
(43) Pub. Date: Sep. 17, 2026

(54) METHOD FOR PREDICTING CHLORITE GROWTH BASED ON MICROBIAL ACTIVITY

(71) Applicant: Chengdu University of Technology, Chengdu (CN)

(72) Inventors: LaiXing Cai, Chengdu (CN); Yuhang Li, Rizhao (CN); Tian Yang, Chengdu (CN); Jingchun Tian, Chengdu (CN); Anqing Chen, Chengdu (CN); Yantao Liao, Chongqing (CN); Jianlian Zhu, Chongqing (CN)

(21) Appl. No.: 19/674,131

(22) Filed: May 12, 2026

(30) Foreign Application Priority Data
Aug. 5, 2025 (CN) 202511091551.8

(51) Int. Cl. G16B 40/20

(52) U.S. Cl. CPC G16B 40/20

(57) ABSTRACT

The present application discloses a method for predicting chlorite growth based on microbial activity. The method includes: S1. generating a comprehensive data set by performing dynamic quantification monitoring of microbial activity and synchronous acquisition of environmental parameters based on a geological environment monitoring system; S2. establishing a kinetic model of microbial influence on chlorite nucleation and growth based on the comprehensive data set and molecular dynamics, and constructing a standardized training data set; S3. constructing a spatiotemporal prediction model of microbial activity based on the standardized training data set; and S4. dynamically updating and performing error correction on the spatiotemporal prediction model of microbial activity to obtain a final spatiotemporal prediction model. When prediction results indicate abnormal changes in chlorite growth rate or crystal morphology, a warning mechanism of the geological environment monitoring system is triggered, and monitoring frequency and sampling strategy are automatically adjusted.

### CROSS-REFERENCE TO RELATED APPLICATIONS

This application claims priority to Chinese Patent Application No. CN202511091551.8, filed on Aug. 5, 2025, which is hereby incorporated by reference in its entirety.

### TECHNICAL FIELD

The present application relates to the technical field of geological environment monitoring and prediction, and in particular, to a method for predicting chlorite growth based on microbial activity.

### BACKGROUND

Chlorite is a common iron-magnesium-aluminum layered silicate mineral in sedimentary environments, and the formation process of chlorite is influenced jointly by physical, chemical, and biological factors. For a long time, research on chlorite growth has mainly focused on physicochemical conditions such as temperature, pH, and solution composition, and the understanding of the genesis and distribution of chlorite has mostly been established based on laboratory synthesis and field investigations. However, recent studies have found that microorganisms play an important role in mineral formation. Some microorganisms can significantly affect the crystal structure, morphology, and formation rate of chlorite by secreting metabolic products, regulating local chemical environments, or mediating crystal nucleation and growth.

Existing monitoring methods mostly adopt a single technical means, and although certain biological activity information can be provided, these methods each suffer from limitations such as a limited application scope, insufficient spatial resolution, or weak dynamic response capability, making it difficult to fully and continuously reflect the actual role of microorganisms in the formation process of chlorite. Meanwhile, most existing chlorite growth models have not fully integrated microorganism-related data, and prediction methods still rely mainly on fitting static parameters, lacking the ability to respond to dynamic changes of microorganisms in actual geological environments.

In view of the foregoing, achieving dynamic monitoring of microbial activity in real geological environments and effectively incorporating the monitoring results into chlorite growth prediction models to improve prediction accuracy and adaptability has become a technical problem urgently to be solved.

### SUMMARY

An objective of the present application is to provide a method for predicting chlorite growth based on microbial activity, which can achieve accurate prediction and dynamic warning of chlorite nucleation, growth kinetics, and crystal morphology by quantifying microbial activity in real time and integrating multi-source data including Raman spectroscopy, electrochemical impedance, fluorescence probes, and environmental parameters, in combination with molecular dynamics simulation and an adaptive deep learning architecture.

To achieve the foregoing purpose, the present application provides the following technical solutions.

In a first aspect, the present application provides a method for predicting chlorite growth based on microbial activity. The method includes:
S1. generating a comprehensive data set by performing dynamic quantification monitoring of microbial activity and synchronous acquisition of environmental parameters based on a geological environment monitoring system, where the comprehensive data set includes microbial activity indicators, environmental parameters, and chlorite growth data;
S2. establishing a kinetic model of microbial influence on chlorite nucleation and growth based on the comprehensive data set and molecular dynamics, and constructing a standardized training data set based on input data and output data of the kinetic model;
S3. constructing a spatiotemporal prediction model of microbial activity based on the standardized training data set; and
S4. dynamically updating and performing error correction on the spatiotemporal prediction model of microbial activity to obtain a final spatiotemporal prediction model, and outputting corrected chlorite growth prediction results based on the final spatiotemporal prediction model.

Optionally, in S1, generating the comprehensive data set by performing dynamic quantification monitoring of microbial activity and synchronous acquisition of environmental parameters based on the geological environment monitoring system is specifically as follows:
the geological environment monitoring system includes a Raman spectroscopy detection module, an electrochemical impedance measurement module, a fluorescence probe labeling module, an environmental parameter monitoring module, and a data fusion analysis module; where
the Raman spectroscopy detection module is configured to identify characteristic peaks of microbial metabolites;
the electrochemical impedance measurement module is configured to detect changes in microbial film resistance;
the fluorescence probe labeling module is configured to label secretion states of extracellular polymers;
the environmental parameter monitoring module is configured to acquire environmental factor data; and
the data fusion analysis module is configured to establish a multi-source data fusion framework by integrating real-time monitoring data, historical geological data, and laboratory analysis results, and to construct a comprehensive data set including microbial activity indicators, environmental parameters, and chlorite growth data through the multi-source data fusion framework.

Optionally, the multi-source data fusion framework is specifically as follows:
the multi-source data fusion framework adopts a hierarchical fusion architecture, including sensor-level fusion, feature-level fusion, and decision-level fusion; where
the sensor-level fusion is configured to perform temporal registration, spatial registration, and data association processing on raw data from sensors of the same type;
the feature-level fusion is configured to extract correlated features and complementary information among different data sources, and to construct a multidimensional feature vector space; and
the decision-level fusion is configured to generate microbial activity evaluation results by combining fusion results from the sensor-level fusion and fusion results from the feature-level fusion.

Optionally, S2 specifically includes:
predicting a complexation reaction mechanism between microbial extracellular polymers and chlorite precursor ions through molecular dynamics simulation, and verifying the prediction through chlorite synthesis experiments under laboratory-controlled conditions to establish a kinetic model of microbial activity on chlorite nucleation and growth,
where the kinetic model is configured to quantify a regulatory effect of microbial metabolites on chlorite crystal morphology and growth direction; and
constructing a standardized training data set based on the comprehensive data set, where the standardized training data set includes temporal features of microbial activity, spatial distribution patterns, and environmental parameter variations, and
the standardized training data set is configured to capture the influence of environmental parameter changes on microbial community succession and a feedback regulatory mechanism of microbial activity fluctuations on chlorite growth patterns.

Optionally, constructing the standardized training data set specifically includes:
organizing the comprehensive data set using a spatiotemporal gridding method, and dividing the comprehensive data set along a temporal dimension to obtain a plurality of time windows, where each time window includes 6 to 24 hours of continuous monitoring data;
establishing a three-dimensional grid in a spatial dimension based on monitoring point locations, where a grid resolution is 1-10 m, and adjusting the three-dimensional grid according to geological complexity of an monitoring area, where
each spatiotemporal grid unit includes microbial activity indicators, environmental parameters, and chlorite growth data.

Optionally, the spatial distribution patterns specifically include:
spatial interpolation, spatial clustering, and spatial autocorrelation analysis; where
the spatial interpolation is configured to generate a continuous spatial distribution map based on microbial activity data from discrete sampling points;
the spatial clustering is configured to identify high-value zones, low-value zones, and transition zones of microbial activity, revealing the spatial distribution pattern of microbial activity; and
the spatial autocorrelation analysis is configured to quantitatively evaluate spatial correlation and spatial heterogeneity of microbial activity.

Optionally, establishing the kinetic model of microbial activity on chlorite nucleation and growth specifically includes:
establishment of a nucleation kinetic model: calculating a nucleation rate equation including biological factors based on classical nucleation theory and non-classical nucleation theory by considering the influence of microbial metabolites on nucleation energy barrier, nucleation rate, and critical nucleus size;
establishment of a growth kinetic model: calculating growth rates of each crystal face of chlorite influenced by microbial extracellular polymers using helical growth mechanisms, two-dimensional nucleation mechanisms, and multi-step growth mechanisms; and
establishment of a morphology control model: predicting morphology evolution of chlorite crystals under microbial activity conditions based on Wulff configuration theory of crystal morphology by considering selective adsorption of organic molecules and surface energy modification effects.

Optionally, constructing the spatiotemporal prediction model of microbial activity based on the standardized training data set specifically includes:
designing an adaptive deep learning architecture integrating a convolutional neural network, a long short-term memory network, and an attention mechanism; and
constructing the spatiotemporal prediction model of microbial activity by combining the standardized training data set with the adaptive deep learning architecture.

Optionally, the adaptive deep learning architecture specifically includes:
a multi-layered structure including an input layer, a feature extraction layer, a feature fusion layer, a temporal modeling layer, and an output layer; where
the input layer is configured to standardize and encode the comprehensive data set and convert different types of data into vector forms processable by a network;
the feature extraction layer is configured to extract local features and global features by using a combination of one-dimensional convolutional neural networks and two-dimensional convolutional neural networks to process temporal data and spatial data, respectively;
the feature fusion layer is configured to adaptively fuse different types of local and global features using attention mechanisms and gating mechanisms, and generate fused features including microbial activity and environmental parameter information;
the temporal modeling layer is configured to model temporal dependencies of microbial activity and environmental parameters based on the fused features output from the feature fusion layer; and
the output layer is configured to output chlorite growth prediction results based on the temporal dependencies using fully connected layers and activation functions.

Optionally, dynamically updating and performing error correction on the spatiotemporal prediction model of microbial activity to obtain the final spatiotemporal prediction model specifically includes:
generating the final spatiotemporal prediction model through dynamic updating and error correction of the spatiotemporal prediction model of microbial activity using Kalman filtering and particle filtering algorithms, including:
when prediction results indicate abnormal changes in chlorite growth rate or crystal morphology, triggering a warning mechanism of the geological environment monitoring system and adjusting monitoring frequency and sampling strategy;
when prediction results fall within a preset growth pattern range, maintaining a current monitoring strategy and continuously performing chlorite growth prediction; and
when prediction results exceed a preset range, increasing monitoring density and recalibrating prediction model parameters until prediction accuracy meets requirements.

According to specific embodiments provided in the present application, the present application achieves the following technical effects:

The present application provides a method for predicting chlorite growth based on microbial activity, and the integrated monitoring system establishes a data foundation for quantitative study of a coupling relationship between microbial activity and environment, which significantly improves spatiotemporal resolution and reliability of the prediction model.

The structured comprehensive data set formed by the multi-source data fusion framework provides data support for constructing the chlorite growth prediction model, which overcomes the limitations of traditional chlorite studies in terms of single data dimension, poor timeliness, and lack of microorganism-related variables.

Through molecular dynamics simulation and experimental verification, the complexation reaction mechanism between microbial extracellular polymers and chlorite precursor ions is investigated, and a kinetic model of chlorite nucleation and growth under microbial regulation is constructed, which significantly advances quantitative understanding of mineral-microbe interaction mechanisms.

Based on the fused multi-source data set, the method captures the influence of environmental changes on microbial community succession and the feedback regulatory mechanism on chlorite growth, which achieves full-process modeling of the complex geochemical process of “environmental disturbance-microbial response-mineral precipitation”.

The chlorite growth prediction model capable of adaptively extracting spatiotemporal features of microbial activity successfully addresses the modeling challenges of geological environment monitoring data, including high noise, multi-scale characteristics, and strong nonlinearity.

In summary, the method for predicting chlorite growth according to the present application systematically constructs an intelligent geological monitoring platform by integrating multiple advanced monitoring technologies, data fusion strategies, and intelligent modeling algorithms, which dynamically senses microbial activity, responds to environmental disturbances, and outputs prediction results in real time.

An organic closed loop is formed among all steps, thereby enhancing real-time performance, accuracy, and adaptability of geological system prediction and overcoming deficiencies of traditional geological prediction methods in recognizing microbial regulatory mechanisms and modeling capability.

### BRIEF DESCRIPTION OF DRAWINGS

To describe the solutions in embodiments of the present application or in the conventional technology more clearly, the following briefly describes the accompanying drawings for describing embodiments. It is clear that the accompanying drawings in the following descriptions show merely some embodiments of the present application, and a person of ordinary skill in the art may still derive other drawings from these accompanying drawings without creative efforts.

FIG. 1 is a flowchart of a method for predicting chlorite growth based on microbial activity according to an embodiment of the present application;

FIG. 2 is a schematic diagram of samples taken according to an embodiment of the present application, where the sampling site on the left is the coastal area of the Yellow Sea at a depth of 57 cm from the surface, and the sampling site on the right is the Yellow River Delta at a depth of 15 cm from the surface;

FIG. 3a is a graph showing a relationship between particle size C value and relative content of illite for the samples shown in FIG. 2;

FIG. 3b is a graph showing a relationship between particle size M value and relative content of illite for the samples shown in FIG. 2;

FIG. 3c is a graph showing a relationship between particle size C value and relative content of chlorite for the samples shown in FIG. 2; and

FIG. 3d is a graph showing a relationship between particle size M value and relative content of chlorite for the samples shown in FIG. 2.

### DESCRIPTION OF EMBODIMENTS

The following clearly and completely describes the technical solutions in embodiments of the present application with reference to the accompanying drawings in embodiments of the present application. It is clear that the described embodiments are merely a part rather than all of embodiments of the present application. Based on the embodiments of the present application, all other embodiments obtained by those of ordinary skill in the art without creative effort fall within the protection scope of the present application.

To make the objectives, features, and advantages of the present invention more apparent and understandable, the following describes the present invention in detail with reference to the accompanying drawings and specific implementations.

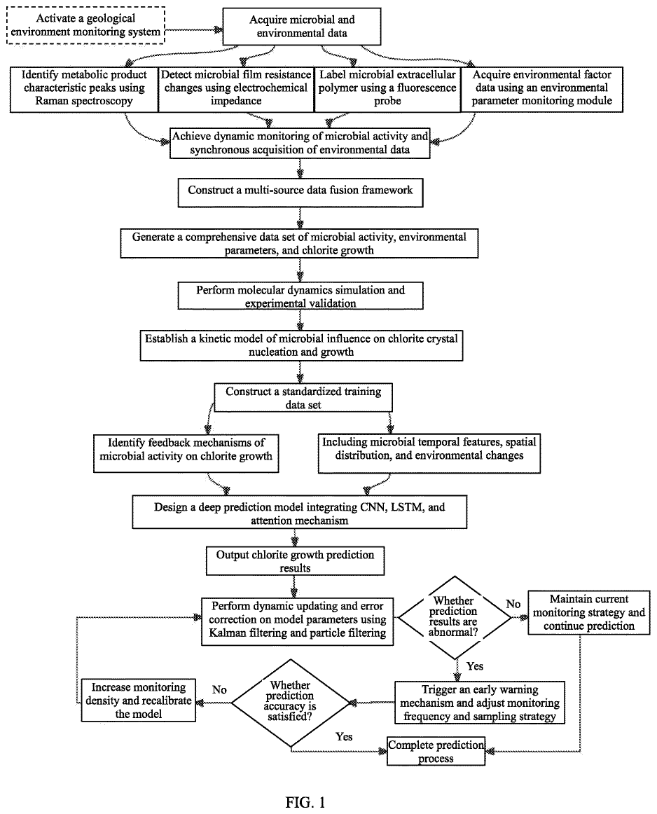

As shown in FIG. 1, an embodiment of the present application provides a method for predicting chlorite growth based on microbial activity. The method includes:
S1. generating a comprehensive data set by performing dynamic quantification monitoring of microbial activity and synchronous acquisition of environmental parameters based on a geological environment monitoring system, where the comprehensive data set includes microbial activity indicators, environmental parameters, and chlorite growth data;
S2. establishing a kinetic model of microbial influence on chlorite nucleation and growth based on the comprehensive data set and molecular dynamics, and constructing a standardized training data set based on input data and output data of the kinetic model;
S3. constructing a spatiotemporal prediction model of microbial activity based on the standardized training data set; and
S4. dynamically updating and performing error correction on the spatiotemporal prediction model of microbial activity to obtain a final spatiotemporal prediction model, and outputting corrected chlorite growth prediction results based on the final spatiotemporal prediction model.

The chlorite growth prediction results obtained through the foregoing steps can be used to guide a plurality of operations during the drilling and completion process. For example,

Guidance for fracturing operations: A fracturing truck is started, and a fracturing fluid is pumped into the formation according to the design requirements of displacement and pump pressure. The proppant-carrying fluid is injected through a high-pressure pump. The displacement fluid is injected through a high-pressure pump to push the proppant-carrying fluid into fractures generated by the fracturing fluid. During the fracturing process, the perforation section is determined based on the chlorite growth prediction results: formation sections with a chlorite volume fraction lower than 5% are selected for perforation, and the perforation orientation is aligned with the direction of the low chlorite development zone, thereby avoiding chlorite-enriched zones.

Guidance for drilling operations: A drilling rig is started, and drilling operations are performed using a drill string and drill bit. The drilling fluid is pumped and circulated to remove rock cuttings generated during drilling. After drilling a certain depth, casing is set and cementing is performed to stabilize the wellbore and prevent collapse. During this process, based on the chlorite growth prediction results, biocides or bactericides are added to the drilling fluid in blocks with high microbial activity at an addition rate of 0.1% to 0.5%. This addition prevents degradation of polymers in the drilling fluid, including organic thickeners and fluid-loss additives.

Optionally, in S1, generating the comprehensive data set by performing dynamic quantification monitoring of microbial activity and synchronous acquisition of environmental parameters based on the geological environment monitoring system is specifically as follows:
the geological environment monitoring system includes a Raman spectroscopy detection module, an electrochemical impedance measurement module, a fluorescence probe labeling module, an environmental parameter monitoring module, and a data fusion analysis module; where
the Raman spectroscopy detection module is configured to identify characteristic peaks of microbial metabolites;
the electrochemical impedance measurement module is configured to detect changes in microbial film resistance;
the fluorescence probe labeling module is configured to label secretion states of extracellular polymers;
the environmental parameter monitoring module is configured to acquire environmental factor data; and
the data fusion analysis module is configured to establish a multi-source data fusion framework by integrating real-time monitoring data, historical geological data, and laboratory analysis results, and to construct a comprehensive data set including microbial activity indicators, environmental parameters, and chlorite growth data through the multi-source data fusion framework.

Specifically, the collaborative integration of the Raman spectroscopy detection module, the electrochemical impedance measurement module, the fluorescence probe labeling module, and the environmental parameter monitoring module achieves multidimensional and dynamic monitoring of microbial activity state and geological environment parameters.

This combined configuration is reflected mainly in two aspects: first, the accuracy of acquiring microbial activity signals is improved; second, the synchronous recording and corresponding analysis of environmental conditions and microbial states are achieved.

Specifically, the Raman spectroscopy detection module can identify Raman characteristic peaks of specific microbial metabolites in real time, thereby facilitating characterization of changes in metabolic activity; the electrochemical impedance measurement module measures changes in biofilm resistance by constructing a three-electrode system, which reflects the variation trend of microbial community structure and adhesion activity; the fluorescence probe labeling module labels and tracks secretion behavior of extracellular polymers, thereby revealing spatial distribution features of reaction potential with mineral precursors; and the environmental parameter monitoring module acquires environmental factor data in real time, including temperature, pH, Eh, and conductivity, thereby providing background support for biogeochemical reactions. This integrated monitoring system establishes a data foundation for quantitative study of a coupling relationship between microbial activity and environment, which significantly improves spatiotemporal resolution and reliability of the prediction model.

Further, the Raman spectroscopy detection module is specifically configured as follows:
a multi-wavelength excitation laser system is adopted, including a 532 nm green laser, a 785 nm near-infrared laser, and a 1064 nm long-wavelength laser, where wavelength switching technology is used to selectively detect different types of microbial metabolites.

A high-sensitivity charge-coupled device detector is provided, which can accurately detect key microbial metabolites at concentrations ranging from 10⁻⁴ to 10⁻⁶ molar.

A surface-enhanced Raman scattering substrate is used to increase the detection signal intensity by one to two orders of magnitude.

A temperature compensation system and a humidity control device are also included, where the working temperature range is −10° C. to 60° C. and the relative humidity range is 20% to 90%, so as to eliminate the influence of environmental factors on spectral measurement accuracy and to achieve accurate reflection of microbial activity state.

In summary, the Raman spectroscopy detection module achieves selective detection of different microbial metabolites through the multi-wavelength excitation system, and accurately identifies metabolites with low abundance in a concentration range of 10⁻⁴ to 10⁻⁸ molar in combination with the high-sensitivity detector, thereby significantly improving detection sensitivity and accuracy. The surface-enhanced Raman scattering technology further amplifies signal intensity by one to two orders of magnitude, which effectively enhances detectability of weak signals. The equipped temperature and humidity control system enables stable operation within −10° C. to 60° C. and 20% to 90% relative humidity, which suppresses external disturbances on spectral accuracy. Overall, these modules achieve efficient and stable monitoring of microbial activity under complex environmental conditions.

Further, the electrochemical impedance measurement module is configured with a three-electrode system, where the working electrode is a platinum electrode or a gold electrode, the reference electrode is a saturated calomel electrode or a silver/silver chloride electrode, and the counter electrode is a platinum wire electrode. The measurement frequency range is set from 0.1 Hz to 10 kHz, and the applied AC voltage amplitude is controlled within a range of 10 to 50 mV. The measurement interval is set to perform a complete impedance spectrum scan every 2 to 6 hours. The parameters, including solution resistance, charge transfer resistance, and double-layer capacitance, are extracted through equivalent circuit fitting.

In summary, the electrochemical impedance measurement module ensures stability of the measurement process and high accuracy of data through the three-electrode configuration. The use of platinum or gold as the working electrode material provides excellent conductivity and biocompatibility, and enhances the electrochemical response of microbial films. The selection of standard electrode systems for the reference and counter electrodes ensures stable potential control. The frequency range of 0.1 Hz to 10 kHz and AC voltage amplitude of 10 to 50 mV covers the impedance characteristics of microbial films from diffusion processes to interfacial processes. Performing impedance spectrum scans every 2 to 6 hours facilitates dynamic tracking of microbial activity. The key parameters are extracted through equivalent circuit fitting, which can accurately reflect the structure and metabolic state of microbial films, thereby providing a reliable basis for quantitative evaluation of microbial activity.

Further, the fluorescence probe labeling module labels extracellular polysaccharides using fluorescein isothiocyanate with an excitation wavelength of 495 nm and an emission wavelength of 519 nm, labels extracellular proteins using Rhodamine B with an excitation wavelength of 550 nm and an emission wavelength of 570 nm, and labels extracellular DNA using anthocyanin with an excitation wavelength of 650 nm and an emission wavelength of 670 nm. Fluorescence detection is performed using a fluorescence microscope, with a spatial resolution of 1 μm and a temporal resolution of one image every 1 to 2 hours. Image processing algorithms are applied to quantitatively analyze the secretion intensity and spatial distribution pattern of each type of extracellular polymer. A standard curve is established between fluorescence intensity and extracellular polymer concentration, and a fluorescence intensity variation threshold of 30% is set to monitor significant changes in microbial secretion activity.

In summary, the fluorescence probe labeling module achieves selective recognition and high-resolution monitoring of different extracellular polymer components with a multi-channel fluorescence labeling strategy by using specific wavelength dyes for extracellular polysaccharides, proteins, and DNA. The combination of fluorescein isothiocyanate, Rhodamine B, and anthocyanin can cover major components of microbial extracellular polymers and significantly improves characterization accuracy of secretion activity. The distribution and intensity variation of extracellular polymers can be dynamically acquired by using a fluorescence microscope with a spatial resolution of 1 μm and a temporal sampling interval of 1 to 2 hours. A standard curve is constructed by analyzing fluorescence intensity by combining an image processing algorithms, and a relationship between fluorescence intensity and actual concentration is quantified. A fluorescence intensity variation threshold of 30% is set, and significant fluctuations in microbial secretion activity can be effectively identified, so that a critical biological basis is provided for predicting an influence of microbial secretion activity on crystal nucleation and growth of chlorite.

Further, the environmental parameter monitoring module includes a temperature sensor array, a pH detection unit, a dissolved oxygen concentration measurement apparatus, an ion concentration detection unit, and a hydrogeological parameter monitoring device, where the temperature sensor array adopts high-precision thermistor sensors to achieve a measurement accuracy of ±0.1° C., a measurement range of −20° C. to 80° C., and a response time of less than 10 seconds; the pH detection unit adopts a composite pH electrode composed of a glass electrode and a reference electrode and equipped with an automatic calibration function and a temperature compensation mechanism, which ensures a measurement accuracy better than ±0.1 pH unit within a range of 2 to 12 pH; the ion concentration detection system integrates selective electrodes for potassium ion, sodium ion, calcium ion, magnesium ion, and silicate ion, has a measurement range of 10⁻⁵ to 10⁻¹ mol/L, and is capable of real-time monitoring of key ion concentration variations related to chlorite formation; and the hydrogeological parameter monitoring device includes a groundwater level monitor with an accuracy of ±1 cm, an osmotic pressure measurement device with a measurement range of 0 to 1000 kPa, and a soil moisture sensor with a measurement range of 0% to 100% volumetric water content, and provides comprehensive environmental parameter background data for microbial activity and chlorite growth.

In summary, the environmental parameter monitoring module achieves comprehensive and real-time monitoring of key physicochemical parameters in the chlorite growth environment by integrating high-precision and multifunctional sensor devices. The temperature sensor array adopts a thermistor with fast response and a measurement accuracy of ±0.1° C., which ensures precise capture of temperature variations, covers a wide range of −20° C. to 80° C., and adapts to complex geological conditions. The pH detection unit is equipped with a composite glass electrode, automatic calibration, and temperature compensation functions, which ensures accurate measurement within a pH range of 2 to 12, and enables dynamic monitoring of acid-base conditions during microbial metabolism and mineral precipitation processes. The ion concentration detection system provides selective electrodes for potassium ion, sodium ion, calcium ion, magnesium ion, and silicate ion, which effectively monitors variations of solution chemical components closely related to crystal nucleation and growth kinetics of chlorite. The hydrogeological parameter device covers three indicators of groundwater level, osmotic pressure, and soil volumetric water content, and reflects the hydrological environment with centimeter-level, kilopascal-level, and percentage volumetric precision, thereby forming a closed-loop environmental background data set for microbial activity and mineral formation. The overall module enables the system to achieve high-resolution synchronous monitoring of environmental factors and biogeological processes, thereby providing scientific and comprehensive environmental support for predicting chlorite growth.

Optionally, the multi-source data fusion framework is specifically as follows:
the multi-source data fusion framework adopts a hierarchical fusion architecture, including sensor-level fusion, feature-level fusion, and decision-level fusion; where
the sensor-level fusion is configured to perform temporal registration, spatial registration, and data association processing on raw data from sensors of the same type;
the feature-level fusion is configured to extract correlated features and complementary information among different data sources, and to construct a multidimensional feature vector space; and
the decision-level fusion is configured to generate microbial activity evaluation results by combining fusion results from the sensor-level fusion and fusion results from the feature-level fusion.

Specifically, a multi-source data fusion framework is established by integrating real-time monitoring data, historical geological data, and laboratory analysis results, which forms a structured comprehensive data set, and provides data support for constructing a chlorite growth prediction model, thereby overcoming the limitations in traditional chlorite studies of single-dimensional data, poor timeliness, and lack of microbial activity-related variables.

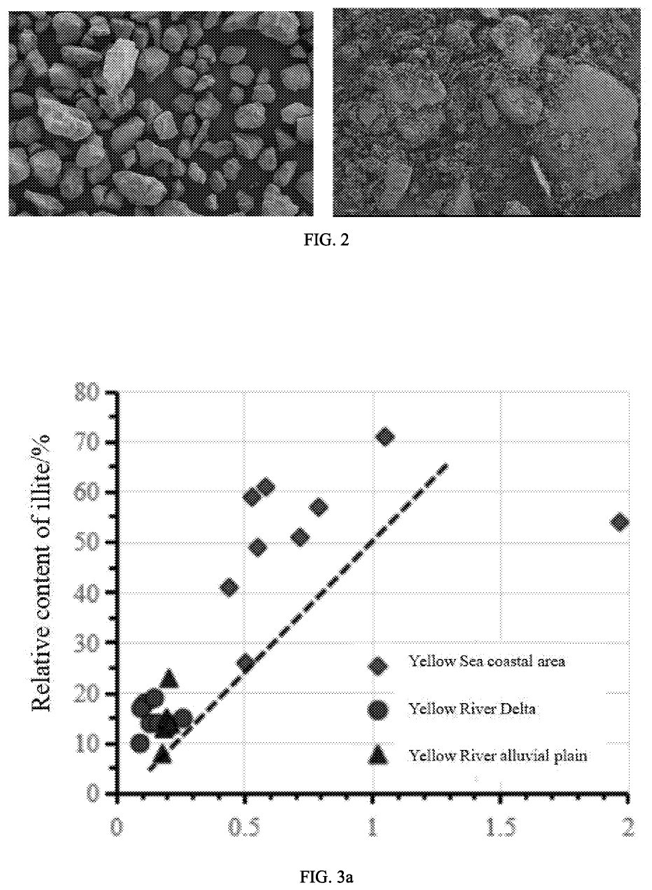

As shown in FIG. 2, samples are simulated and collected from the Badain Jaran Desert in deltaic and coastal environments. The sampling site on the left is the coastal area of the Yellow Sea at a depth of 57 cm from the surface, and the sampling site on the right is the Yellow River Delta at a depth of 15 cm from the surface.

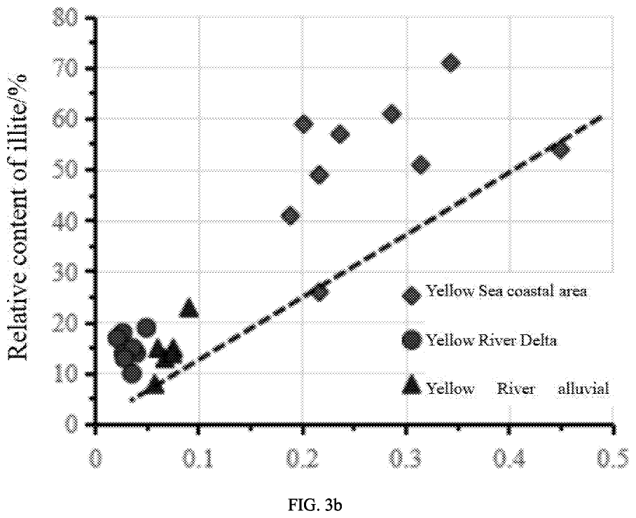

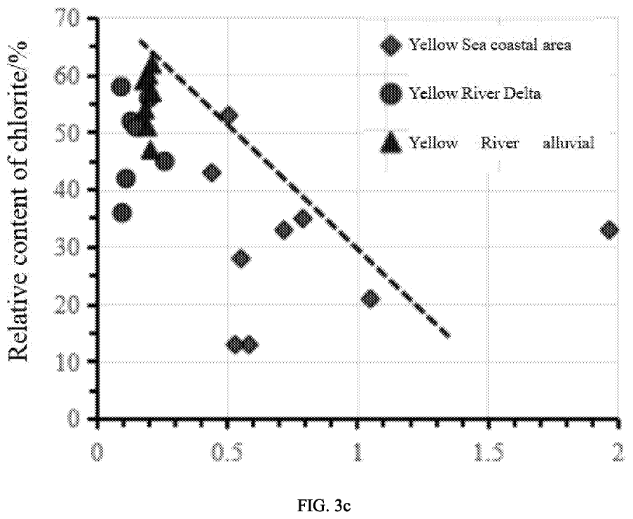

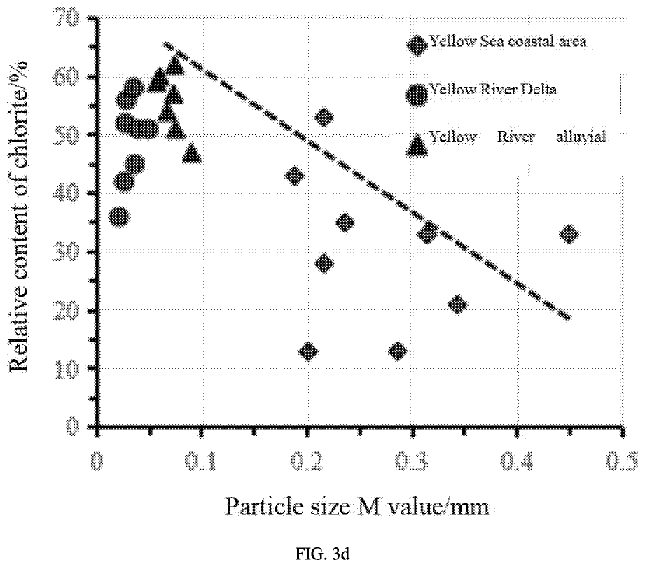

As shown in FIGS. 3a to 3d, analyses are performed based on the simulated Badain Jaran Desert samples, and corresponding relationship diagrams are established. The C value represents the median particle size, d50, and the M value represents the particle size corresponding to 1% on the cumulative curve, d1. Correlation between depositional environment and clay minerals: coarse particles, high-energy conditions, and oxidative environments are conducive to illite preservation, and low-energy, fine particles, and reductive environments promote chlorite formation. Illite possesses a stable crystal structure and slightly higher hydraulic equivalence, which allows co-deposition with coarse particles.

Reductive environments facilitate Fe²⁺ transformation, which provides necessary conditions for chlorite formation.

The fusion framework effectively integrates dynamically acquired microbial activity indicators, on-site environmental parameters, historical mineral formation data, and chlorite formation parameters under controlled experimental conditions and constructs a multi-scale, cross-domain data platform, thereby achieving continuous modeling from macroscopic environmental context to microscopic reaction mechanisms.

Specifically, the platform may enable attribution analysis of the relationships between microbial metabolic activity and mineral phase transitions under different geological scenarios, reveal co-evolution pathways, and support preprocessing operations including feature selection, variable normalization, and data gap imputation, thereby providing highly cleaned training data for subsequent model training and parameter optimization.

Further, the data fusion analysis module adopts a multi-level data fusion architecture, including a data preprocessing layer, a feature extraction layer, an information fusion layer, and a decision output layer, where the data preprocessing layer is configured to perform time synchronization, noise filtering, missing value imputation, and outlier detection on raw data from each detection module, to ensure data quality and consistency; the feature extraction layer is configured to extract key feature parameters from multi-dimensional detection data that reflect microbial activity and environmental variations; the information fusion layer is configured to perform weighted integration of information from different detection modules to construct fusion indicators comprehensively reflecting microbial activity status; and the decision output layer generates microbial activity assessment reports based on the fusion results, including activity level classification, trend prediction, and abnormal state identification, and provides reliable microbial activity input parameters for chlorite growth prediction.

In summary, the data fusion analysis module achieves efficient integration and intelligent interpretation of multi-source heterogeneous data through the multi-level architecture, thereby significantly enhancing the accuracy and reliability of microbial activity monitoring. First, the data preprocessing layer ensures temporal alignment of data from each module through time synchronization, and performs noise filtering, missing value completion, and outlier detection, thereby effectively enhancing the integrity and quality of input data and preventing misjudgment caused by data anomalies. Second, the feature extraction layer identifies key indicators closely related to microbial activity and environmental variations from multi-dimensional monitoring data, such as electrochemical impedance feature parameters, fluorescence intensity variations, and environmental factor fluctuation characteristics, thereby achieving information refinement and dimensionality reduction. The information fusion layer integrates multi-module features based on a weighted algorithm to form fusion indicators reflecting the overall microbial activity status, thereby overcoming the limitations of single detection methods and enhancing sensitivity to complex environmental changes. Finally, the decision output layer classifies activity levels, predicts trends, and identifies abnormal states based on the fusion results, and generates structured assessment reports, thereby providing precise and dynamic input data support for subsequent chlorite growth prediction.

Optionally, S2 specifically includes:
predicting a complexation reaction mechanism between microbial extracellular polymers and chlorite precursor ions through molecular dynamics simulation, and verifying the prediction through chlorite synthesis experiments under laboratory-controlled conditions to establish a kinetic model of microbial activity on chlorite nucleation and growth,
where the kinetic model is configured to quantify an regulatory effect of microbial metabolites on chlorite crystal morphology and growth direction; and
constructing a standardized training data set based on the comprehensive data set, where the standardized training data set includes temporal features of microbial activity, spatial distribution patterns, and environmental parameter variations, and
the standardized training data set is configured to capture the influence of environmental parameter changes on microbial community succession and a feedback regulatory mechanism of microbial activity fluctuations on chlorite growth patterns.

Specifically, through molecular dynamics simulation and experimental verification, the complexation reaction mechanism between microbial extracellular polymers and chlorite precursor ions is investigated, and a kinetic model of chlorite nucleation and growth under microbial regulation is constructed, which significantly advances quantitative understanding of mineral-microbe interaction mechanisms. The advancement is manifested in two aspects. In one aspect, molecular dynamics simulation can reveal, at the atomic scale, the binding modes and energy barriers of specific extracellular polymer functional groups with precursor ions such as Fe²⁺ and Mg²⁺, and clarify nucleation site selection and preferred crystallization orientation. In another aspect, controlled experiments simulate microbial activity regulation conditions, such as carbon source concentration and redox state, to investigate effects on chlorite morphology and growth rate, thereby achieving mutual verification between theoretical simulation and actual processes.

Based on the integrated multi-source data set, a standardized training data set is constructed, including microbial activity temporal features, spatial distribution patterns, and environmental parameter variations, thereby capturing the influence of environmental changes on microbial community succession and the feedback regulation of environmental changes on chlorite growth. The advancement lies in achieving full-process modeling of the complex geochemical process of “environmental perturbation-microbial response-mineral precipitation”. By introducing spatial distribution features and temporal evolution paths, dynamic succession patterns of microbial activity under varying environmental factors (such as dramatic fluctuations in pH and temperature) can be identified. In combination with chlorite deposition rate information, time-lag characteristics and nonlinear feedback of the existing biogeochemical coupling process are inferred.

Further, the molecular dynamics simulation adopts the GROMACS or LAMMPS software platform and selects CHARMM or AMBER force field parameters. The simulation system includes 1000 to 5000 atoms, with simulation times ranging from 5 to 50 nanoseconds and time steps of 1 to 2 femtoseconds. Temperature is controlled within 25° C. to 50° C., and isothermal or isothermal-isobaric simulations are conducted using NVT or NPT ensembles. The coordination structures between microbial extracellular polymer and metal ions are analyzed by radial distribution function, average interaction energies and diffusion coefficients are calculated, intermolecular interaction potential functions are established, and dynamic processes and mechanisms of complexation reactions are revealed through molecular dynamics trajectory analysis.

In summary, the molecular dynamics simulation precisely simulates molecular interactions between microbial extracellular polymer and metal ions by using the GROMACS or LAMMPS platform in combination with CHARMM or AMBER force field parameters. The simulation system includes 1000 to 5000 atoms and a time span of 5 to 50 nanoseconds, ensuring sufficient spatial and temporal resolution, with a time step of 1 to 2 femtoseconds to maintain numerical stability. The temperature is controlled within 25° C. to 50° C., and NVT or NPT ensembles are used to reproduce physical conditions under isothermal or isothermal-isobaric environments. Radial distribution function analysis is used to accurately describe coordination structures between extracellular polymer and metal ions, calculate average interaction energies and diffusion coefficients, and reveal intermolecular binding strength and dynamic behavior. Intermolecular interaction potential functions constructed based on simulation trajectories are used to analyze the dynamic evolution process and mechanism of complexation reactions, which provides microscopic theoretical support for microbial regulation of chlorite nucleation and growth.

Further, the microbial metabolite characteristic peak identification process includes the following steps. First, automatic baseline correction of spectral data is performed by combining polynomial fitting with asymmetric least squares, to effectively remove fluorescence background and baseline drift. Subsequently, peak boundary identification is performed on the corrected spectral data using the second derivative method and continuous wavelet transform, followed by peak shape fitting using Gaussian functions. Finally, peak areas are calculated by numerical integration to quantitatively reflect microbial metabolite content. Next, based on a database including standard Raman spectra of 200 to 300 common microbial metabolites, spectral matching algorithms are applied to the calculated peak areas and their characteristics to achieve rapid metabolite identification.

In summary, high-precision spectral baseline correction is achieved by introducing polynomial fitting combined with asymmetric least squares, peak position identification is performed using the second derivative method and continuous wavelet transform, and quantitative analysis of metabolite content is conducted using Gaussian fitting and numerical integration. Consequently, rapid and accurate metabolite identification is achieved through spectral matching with the standard database, thereby significantly enhancing the automation, quantitative accuracy, and identification efficiency of microbial metabolite analysis.

Further, during the microbial membrane resistance variation detection process, impedance spectroscopy measurement is first performed using a frequency scan method with a sinusoidal excitation signal to obtain the impedance response characteristics of the microbial membrane at different frequencies. Subsequently, based on the measured impedance spectra, an equivalent circuit model including solution resistance, charge transfer resistance, double-layer capacitance, and Warburg diffusion impedance is used to fit the impedance data, so as to accurately describe the electrochemical behavior of the microbial membrane-electrode interface. Finally, a nonlinear least squares fitting algorithm is applied to the fitted results to extract membrane resistance, membrane capacitance, ion transport impedance, and reaction kinetics parameters.

The equivalent circuit model processing procedure specifically includes the following steps. First, the electrochemical behavior of the microbial membrane-electrode interface is abstracted as a circuit network including solution resistance, charge transfer resistance, double-layer capacitance, and Warburg diffusion impedance. Second, the impedance spectra are fitted to estimate parameters corresponding to each circuit element according to the physical significance of the circuit element: solution resistance reflects the conductivity of the electrolyte, appearing in the real part of high-frequency impedance; charge transfer resistance corresponds to the impedance of electron transfer reactions occurring at the electrode surface, primarily affecting mid-frequency impedance; double-layer capacitance simulates the capacitive characteristics of the electrode interface charge layer, affecting the imaginary component of impedance; and Warburg diffusion impedance describes the ion diffusion process near the electrode surface, appearing as frequency-dependent characteristics in low-frequency impedance. Finally, nonlinear least squares fitting is used to fit the real and imaginary parts of the impedance spectra to the equivalent circuit model, determining the parameter values of each element and achieving quantitative analysis of microbial membrane resistance variation.

The reaction kinetics parameters include complexation reaction rate constant, reflecting the speed of complex formation; coordination number and type of complexing ligand, affecting complex stability; diffusion coefficient, affecting the encounter frequency of ions and polymer molecules; and surface adsorption constant, describing regulation of the crystal growth surface by extracellular polymer.

In summary, by measuring the microbial membrane impedance spectra using a frequency scan method, fitting the impedance data with an equivalent circuit model including a plurality of electrochemical elements, and extracting key electrochemical parameters by using a nonlinear least squares algorithm, precise modeling of microbial membrane-electrode interface behavior and efficient extraction of membrane resistance and related parameters are achieved, thereby improving the accuracy, resolution, and quantitative capability of microbial membrane electrochemical characteristic detection.

Further, the standardized training data set constructed from the multi-source data fusion results is organized through a spatiotemporal gridding method. The data set is divided into different time windows according to the temporal dimension, and each time window includes continuous monitoring data for 6 to 24 hours. The spatial dimension establishes a three-dimensional grid based on monitoring point locations, with a grid resolution of 1 to 10 meters, and is adjusted according to the geological complexity of the monitored area. Each spatiotemporal grid unit includes complete records of microbial activity indicators, environmental parameters, and chlorite growth data. The data standardization process adopts Z-score standardization or min-max standardization to ensure comparability among data of different dimensions.

In summary, the established multi-source data fusion standardized training data set is systematically organized through the spatiotemporal gridding method, which significantly improves data structuring and analysis efficiency. In the temporal dimension, data is divided into continuous time windows of 6 to 24 hours to ensure capture of the dynamic evolution of microbial activity and environmental parameters. In the spatial dimension, a three-dimensional grid is established based on monitoring point locations, with a resolution between 1 and 10 meters, and is adjusted flexibly according to geological complexity to refine spatial heterogeneity representation. Each spatiotemporal unit contains comprehensive data of microbial activity indicators, environmental factors, and chlorite growth, thereby forming a multidimensional coupled feature set. Z-score standardization or min-max standardization is applied to eliminate dimensional differences among variables, thereby enhancing data consistency and comparability.

Further, the multi-source data fusion framework adopts a hierarchical fusion architecture, including sensor-level fusion, feature-level fusion, and decision-level fusion. The sensor-level fusion is configured to perform temporal alignment, spatial alignment, and data association processing on raw data from the same type of sensors to improve spatiotemporal consistency of the data. The feature-level fusion is configured to extract associated features and complementary information among different data sources to construct a multidimensional feature vector space. The decision-level fusion is configured to integrate fusion results from different layers to generate a unified microbial activity assessment. The comprehensive decision weights the indicators based on assigned weights by receiving multidimensional features extracted by feature-level fusion, calculates an overall activity score using fuzzy comprehensive evaluation or machine learning algorithms, further determines the microbial activity state based on thresholds, and finally outputs a unified assessment result, thereby achieving integrated decision-making for multi-source information.

In summary, the multi-source data fusion framework effectively enhances spatiotemporal consistency and information representation through sensor-level fusion, feature-level fusion, and decision-level fusion. The sensor-level fusion achieves temporal and spatial alignment of same-type data, and ensures accurate registration of raw data. The feature-level fusion mines associated features and complementary information among different data sources, to construct a multidimensional feature space and enhance data expressiveness. The decision-level fusion integrates results from all layers to achieve unified and precise microbial activity assessment, thereby significantly improving monitoring accuracy and decision-making reliability.

Further, in the chlorite synthesis experiment, a reaction system is prepared by precisely controlling the stoichiometric ratios, using high-purity FeCl₃·6H₂O, MgCl₂·6H₂O, Al₂ (SO₄)₃·18H₂O, and Na₂SiO₃·9H₂O to simulate the ionic composition in a natural geological environment, and maintain the pH of the reaction system within a range of 5.5 to 8.5. Subsequently, a reaction kettle with temperature control and stirring functions is based to implement temperature regulation, atmospheric pressure reaction, pH adjustment, and reaction time control, thereby precisely managing the reaction conditions. Thereafter, the products are characterized through X-ray diffraction, scanning electron microscopy, transmission electron microscopy, and energy-dispersive X-ray spectroscopy to comprehensively analyze the crystal structure, crystal morphology, and chemical composition of chlorite. Finally, based on time-resolved sampling and quantitative XRD analysis data, a quantitative relationship between chlorite growth rate and reaction conditions is established, which encompasses conversion rate, crystallinity, and crystal size parameters.

In summary, the chlorite synthesis experiment achieves successful simulation of natural geological environment conditions by precisely preparing high-purity metal salt precursors and strictly controlling the ionic composition and pH of the reaction system. The precise adjustment of reaction conditions, including temperature, atmospheric pressure, pH, and reaction time, enables controllability of the chlorite synthesis process. The products are fully characterized through a plurality of advanced techniques, which reveals crystal structure, morphology, and chemical composition, thereby ensuring accurate analysis of synthesized chlorite. Kinetic analysis, in combination with time-resolved sampling and quantitative X-ray diffraction, establishes a quantitative relationship between growth rate and reaction parameters, and provides a scientific basis for understanding the chlorite formation mechanism and optimizing synthesis conditions.

Optionally, constructing the standardized training data set specifically includes:
organizing the comprehensive data set using a spatiotemporal gridding method, and dividing the comprehensive data set along a temporal dimension to obtain a plurality of time windows, where each time window includes 6 to 24 hours of continuous monitoring data;
establishing a three-dimensional grid in a spatial dimension based on monitoring point locations, where a grid resolution is 1-10 m, and adjusting the three-dimensional grid according to geological complexity of an monitoring area, where
each spatiotemporal grid unit includes microbial activity indicators, environmental parameters, and chlorite growth data.

Optionally, geological complexity is quantified through a multi-factor comprehensive assessment method, primarily considering geological factors closely related to the chlorite growth environment, including structural complexity, lithological complexity, and hydrogeological complexity. Structural complexity is evaluated based on fault density, fold development degree, and variation of the structural stress field, with values ranging from 0.1 to 1.0, which reflects the influence of geological structures on microbial habitats and ion migration pathways. Lithological complexity is calculated based on rock type distribution, permeability differences, and chemical composition variability, with values ranging from 0.1 to 0.9, which represents the control of different lithologies on microbial metabolic activity and supply of chlorite precursor ions. Hydrogeological complexity is calculated by combining groundwater flow variations, permeability distribution, and groundwater chemical zoning, with values ranging from 0.1 to 0.8, which quantifies the regulatory effect of hydrogeological conditions on microbial activity and ion transport. A comprehensive complexity index (GCI) is calculated by weighted fusion of the three components, with weight allocation of 0.4 for structural complexity, 0.35 for lithological complexity, and 0.25 for hydrogeological complexity.

Optionally, the three-dimensional grid is adjusted using a hierarchical refinement strategy based on GCI values: a coarse grid resolution of 8 to 10 meters is used when GCI≤0.35, a medium resolution of 4 to 6 meters is used when 0.35<GCI≤0.65, and a fine resolution of 1 to 3 meters is used when GCI>0.65. The specific adjustment process is as follows: first, the average GCI value and spatial gradient for each initial grid unit are calculated; when GCI exceeds the hierarchical threshold or the gradient is greater than 0.2, octree subdivision is triggered to decompose the grid unit into eight sub-grids, thereby doubling the resolution; a buffer transition zone with a width of 0.5 to 1.0 times the coarse grid size is established at boundaries between different resolutions, and gradient interpolation is applied to smoothly connect grid densities; grid density is locally optimized based on monitoring point distribution, with dense grids in areas of concentrated monitoring points and coarse grids in sparse areas, and sub-grids inherit and refine geological attributes through Kriging interpolation to ensure consistency of grid properties with actual geological conditions and monitoring data.

Strict quality control inspection is performed on the adjusted three-dimensional grid. Regarding grid geometry, the aspect ratio of grid units is maintained between 1:1 and 2.5:1, the size ratio of adjacent grids does not exceed 1.5:1, and interior angles of grid units are maintained between 45° and 135°. Regarding grid coverage completeness, all monitoring points are included within grid units, and grid boundaries are consistent with monitored area boundaries. Regarding data consistency, the fit between grid attribute values and measured data is verified, requiring interpolation errors to be less than 15% of measured values, and continuity of attribute values between adjacent grids is checked to avoid unreasonable abrupt changes. Grid units failing quality inspection are readjusted for resolution or re-interpolated for attributes until all quality criteria are satisfied.

Optionally, the spatial distribution patterns specifically include:
spatial interpolation, spatial clustering, and spatial autocorrelation analysis; where
the spatial interpolation is configured to generate a continuous spatial distribution map based on microbial activity data from discrete sampling points;
the spatial clustering is configured to identify high-value zones, low-value zones, and transition zones of microbial activity, revealing the spatial distribution pattern of microbial activity; and
the spatial autocorrelation analysis is configured to quantitatively evaluate spatial correlation and spatial heterogeneity of microbial activity.

Optionally, establishing the kinetic model of microbial activity on chlorite nucleation and growth specifically includes:
establishment of a nucleation kinetic model: calculating a nucleation rate equation including biological factors based on classical nucleation theory and non-classical nucleation theory by considering the influence of microbial metabolites on nucleation energy barrier, nucleation rate, and critical nucleus size;
establishment of a growth kinetic model: calculating growth rates of each crystal face of chlorite influenced by microbial extracellular polymers using helical growth mechanisms, two-dimensional nucleation mechanisms, and multi-step growth mechanisms; and
establishment of a morphology control model: predicting morphology evolution of chlorite crystals under microbial activity conditions based on Wulff configuration theory of crystal morphology by considering selective adsorption of organic molecules and surface energy modification effects.

Further, the kinetic model of microbial activity on chlorite nucleation and growth includes three sub-models: a nucleation kinetic model, a growth kinetic model, and a morphology control model.

The nucleation kinetic model establishes a nucleation rate equation including biological factors based on classical nucleation theory and non-classical nucleation theory by considering the influence of microbial metabolites on nucleation energy barrier, nucleation rate, and critical nucleus size.

The growth kinetic model describes the regulatory effect of microbial extracellular polymer on growth rates of different crystal faces of chlorite crystal by using a spiral growth mechanism, a two-dimensional nucleation mechanism, and a multi-step growth mechanism.

The morphology control model predicts morphology evolution of chlorite crystals under microbial activity conditions based on Wulff configuration theory of crystal morphology by considering selective adsorption of organic molecules and surface energy modification effects.

The nucleation kinetic model is expressed by the following equation:

$$J = {J}_{0} exp ( - \frac{Δ {G}^{*}}{{k}_{B} T} ) \cdot f ( {C}_{bio ) ,}$$

where J₀ represents a pre-exponential factor depending on nucleation site density and diffusion rate; ΔG* represents the nucleation energy barrier; kg represents the Boltzmann constant; T is temperature; f(C_{bio}) represents a correction function representing promotion or inhibition of nucleation by microbial metabolic products; and J represents the nucleation rate, i.e., the number of new nuclei formed per unit volume or unit area per unit time, which reflects the rate of transition of crystals from a disordered state to an ordered state.

The growth kinetic model is expressed by the following equation: Rᵢ=kᵢ(C−C_{eq})·exp(−βᵢθ_{bio}), where kᵢ is the growth rate constant of the crystal face i; C is the concentration of precursor ions in solution; C_{eq} is the equilibrium concentration; βᵢ is the inhibition coefficient of microbial extracellular polymer on the growth of the crystal face i; θ_{bio} represents coverage or binding strength of the extracellular polymer; and Rᵢ represents the growth rate of the i-th crystal face, i.e., the increase in length along the normal direction per unit time, which reflects growth rate along different crystal face directions.

The morphology control model is expressed by the following equation:

$$min {\sum }_{i} {γ}_{i}^{′} {A}_{i} = min {\sum }_{i} ( {γ}_{i} - {Δγ}_{i} ) {A}_{i} ;$$

where γᵢ is the original surface free energy of the crystal face i; Δγᵢ is the surface energy reduction caused by adsorption of microbial metabolic products or extracellular polymer;

$${γ}_{i}^{′} = {γ}_{i} - {Δγ}_{i}$$

is the corrected surface free energy; and Aᵢ is the area of the crystal face i.

In summary, the kinetic model of microbial activity on chlorite nucleation and growth, including the nucleation kinetic model, the growth kinetic model, and the morphology control model, achieves a systematic description of microbial influence on the entire mineral formation process. The nucleation kinetic model, combining classical and non-classical theories, quantifies the influence of microbial metabolic products on nucleation energy barriers, nucleation rates, and critical nucleus sizes, and clarifies the key role of biological factors in early mineral formation. The growth kinetic model introduces spiral growth, two-dimensional nucleation, and multi-step growth mechanisms, reveals the regulatory effect of microbial extracellular polymer on growth rates of different crystal faces, and reflects fine microbial control over crystal structure. The morphology control model, based on the Wulff configuration, considers adsorption of organic molecules and surface energy modification, predicts the morphological evolution of chlorite crystals under microbial activity conditions, and provides theoretical support for understanding microbial regulation of mineral crystal morphology, thereby substantially enhancing quantitative analysis of chlorite growth mechanisms.

Optionally, a spatiotemporal prediction model of microbial activity is constructed based on the standardized training data set, specifically including:
designing an adaptive deep learning architecture integrating a convolutional neural network, a long short-term memory network, and an attention mechanism; and
constructing the spatiotemporal prediction model of microbial activity by combining the standardized training data set with the adaptive deep learning architecture.

Specifically, a deep learning architecture integrating a convolutional neural network (CNN), a long short-term memory network (LSTM), and an attention mechanism is designed to construct a chlorite growth prediction model capable of adaptively extracting spatiotemporal features of microbial activity. The technical advancement lies in successfully addressing modeling challenges associated with geological environment monitoring data, including high noise, multi-scale variability, and strong nonlinearity. The CNN module is mainly configured to identify spatial distribution patterns of microbial activity and environmental parameters. The LSTM module is configured to capture temporal evolution trends of microbial activity. The attention mechanism enhances model response to key variables and abrupt changes, thereby improving model interpretability and prediction accuracy. In addition, data augmentation, transfer learning, and cross-validation are used to enhance model robustness and generalization across different geological conditions. The model predicts growth rates and spatial distributions of chlorite, and further infers morphological evolution of chlorite under different microbial community dominance mechanisms, thereby providing technical support for geological hazard warning and mineral resource assessment.

Optionally, the adaptive deep learning architecture specifically includes:
a multi-layered structure including an input layer, a feature extraction layer, a feature fusion layer, a temporal modeling layer, and an output layer; where
the input layer is configured to standardize and encode the comprehensive data set and convert different types of data into vector forms processable by a network;
the feature extraction layer is configured to extract local features and global features by using a combination of one-dimensional convolutional neural networks and two-dimensional convolutional neural networks to process temporal data and spatial data, respectively;
the feature fusion layer is configured to adaptively fuse different types of local and global features using attention mechanisms and gating mechanisms, and generate fused features including microbial activity and environmental parameter information;
the temporal modeling layer is configured to model temporal dependencies of microbial activity and environmental parameters based on the fused features output from the feature fusion layer; and
the output layer is configured to output chlorite growth prediction results based on the temporal dependencies using fully connected layers and activation functions.

Further, the temporal feature extraction of microbial activity includes three steps: time series preprocessing, feature parameter calculation, and pattern recognition. The time series preprocessing is configured to eliminate the influence of measurement noise and outliers. The feature parameter calculation is configured to extract statistical features, time-domain features, and frequency-domain features. The pattern recognition is configured to identify periodic variations, abrupt variations, and trend variations of microbial activity. The feature extraction process further includes multi-scale analysis, which captures variations of microbial activity at different temporal scales through feature extraction using different time windows.

In summary, the temporal feature extraction of microbial activity captures dynamic variations comprehensively through time series preprocessing, feature parameter calculation, and pattern recognition. The preprocessing step effectively removes noise and outliers to ensure data quality. The feature parameter calculation enriches feature representation through statistical, time-domain, and frequency-domain indices. The pattern recognition reveals periodic, abrupt, and trend variations, reflecting complex microbial dynamics. Meanwhile, the multi-scale analysis captures variations at different temporal scales through feature extraction in a plurality of time windows, which enhances the capability to analyze spatiotemporal evolution of microbial activity.

Further, the spatial distribution pattern analysis includes three components: spatial interpolation, spatial clustering, and spatial autocorrelation analysis. The spatial interpolation generates a continuous spatial distribution map from microbial activity data at discrete sampling points. The spatial clustering identifies high-value, low-value, and transition zones of microbial activity, revealing spatial distribution patterns. The spatial autocorrelation analysis quantitatively evaluates spatial correlation and spatial heterogeneity of microbial activity.

In summary, the spatial distribution pattern analysis enables fine characterization of microbial activity in space through spatial interpolation, clustering, and autocorrelation analysis. The spatial interpolation transforms discrete sampling point data into continuous distribution maps, visually reflecting variation trends. The spatial clustering identifies high-value, low-value, and transition zones, revealing spatial structure characteristics of microbial activity. The spatial autocorrelation analysis quantitatively evaluates spatial correlation and heterogeneity, providing a scientific basis for environmental regulation and chlorite growth prediction.

Further, capturing the feedback regulation mechanism of microbial activity fluctuations on chlorite growth patterns includes the following: Initial molecular structures are constructed using Avogadro based on the known main components of microbial extracellular polymer and the chemical structures of chlorite precursor ions, and microbial extracellular polymer fragments are assigned charges and parameterized with force fields. TIP3P water molecules are added around the initial molecular structures to form a buffer layer of at least 1.5 nm, and sodium and chloride ions are added according to experimental pH conditions to achieve electrical neutrality. Energy minimization is performed to remove unreasonable atomic overlaps, and the system is gradually heated to the target temperature under an isochoric and isothermal ensemble. Subsequently, pressure equilibrium is conducted under an isothermal-isobaric ensemble for at least 1 ns to stabilize system density, temperature, and energy. Thereafter, the system continues to run under an isochoric and isothermal ensemble for 1 ns to eliminate the influence of temperature fluctuations on molecular conformations. Production simulation is performed for at least 100 ns using a 2 fs time step, and trajectory files are recorded every 2 ps. Trajectory files are analyzed to calculate coordination numbers, coordination bond length distributions, and radial distribution functions, observe dynamic interactions between metal ions and microbial extracellular polymer functional groups, and describe complexation reaction pathways from both geometric and energetic perspectives. The binding free energy calculations are performed for selected key frames from the production simulation, energy contributions are decomposed, and the influence of different functional groups on complex stability is quantified to determine dominant complexation sites.

In summary, by combining molecular modeling with molecular dynamics simulation, an interaction model between microbial extracellular polymer and chlorite precursor ions is constructed, which accurately reproduces structural evolution of the system in aqueous solution under specific pH conditions. Trajectory analysis and binding free energy calculations provide an in-depth understanding of dynamic complexation behavior and stability contributions of metal ions with microbial functional groups, thereby achieving precise capture and quantitative analysis of the regulatory mechanism of microbial activity fluctuations on chlorite growth.

Optionally, dynamically updating and performing error correction on the spatiotemporal prediction model of microbial activity to obtain the final spatiotemporal prediction model specifically includes:
generating the final spatiotemporal prediction model through dynamic updating and error correction of the spatiotemporal prediction model of microbial activity using Kalman filtering and particle filtering algorithms, including:
when prediction results indicate abnormal changes in chlorite growth rate or crystal morphology, triggering a warning mechanism of the geological environment monitoring system and adjusting monitoring frequency and sampling strategy;
when prediction results fall within a preset growth pattern range, maintaining a current monitoring strategy and continuously performing chlorite growth prediction; and
when prediction results exceed a preset range, increasing monitoring density and recalibrating prediction model parameters until prediction accuracy meets requirements.

Specifically, by introducing Kalman filtering and particle filtering algorithms, dynamic parameter updating and error adaptive correction of the model are achieved under real-time monitoring conditions, thereby implementing “closed-loop control” of the prediction model and enhancing real-time responsiveness and robustness. During model operation, abrupt changes in environmental data and microbial activity indicators may cause prediction results to deviate from actual trends. The filtering algorithms can reevaluate model states and optimize parameters based on the latest observations, and correct prediction deviations in a timely manner, thereby ensuring continuity and accuracy of results. Particularly, in cases of abnormal chlorite growth rates or drastic changes in crystal morphology, the dynamic correction mechanism can effectively prevent error propagation, and enhance online learning capability and stability of the model, thereby supporting implementation of a high-precision, low-latency geological environment prediction system.

When chlorite growth rates or crystal morphology deviate from expected trends, an early warning mechanism is automatically triggered, and monitoring frequency and sampling strategies are adjusted to optimize resource allocation and improve response efficiency. This enhances the capability to respond to sudden geological events, allows increased data acquisition frequency, and focuses monitoring on abnormal zones and critical indicator variables. If prediction results remain within preset safety ranges, conventional monitoring density is maintained (The monitoring density includes temporal monitoring density and spatial monitoring density. The temporal monitoring density refers to the number of data acquisitions per unit time, and the spatial monitoring density refers to the number of monitoring points per unit area. According to the type of prediction anomaly, temporal monitoring density and/or spatial monitoring density are adaptively increased to enhance monitoring accuracy and reliability of microbial activity and chlorite growth dynamics), thereby reducing energy consumption and data processing burden. In addition, if continuous assessment indicates that prediction results are inconsistent with actual observations, model retraining or parameter recalibration can be further automatically triggered to achieve “model-monitoring-feedback” closed-loop optimization, thereby ensuring long-term stability and prediction accuracy.

In summary, the adaptive deep learning architecture efficiently processes and fuses the comprehensive data set through a multi-level structure to achieve accurate prediction of chlorite growth. The input layer standardizes and encodes different types of data to ensure consistency. The feature extraction layer captures temporal and spatial features by combining one-dimensional and two-dimensional convolutional neural networks to extract local and global information. The feature fusion layer integrates key features adaptively through attention and gating mechanisms, to enhance important information and suppress redundant information. The temporal modeling layer further explores temporal dependencies of microbial activity and environmental parameters. The output layer predicts growth rates, directions, and crystal morphology parameters accurately through fully connected layers and activation functions, to significantly improve prediction precision and reliability.

Further, the convolutional neural network adopts a ResNet-18 or ResNet-34 architecture, including 18 or 34 convolutional layers. Each convolutional layer uses a 3×3 or 5×5 convolution kernel, and ReLU or Leaky ReLU activation functions are used. The training is accelerated by Batch normalization, and gradient vanishing is mitigated by residual connections. The network input layer receives feature images of 64×64 or 128×128 pixels, spatial features are extracted through a plurality of convolutional and pooling layers, and feature vectors are output through fully connected layers. Network training is performed by using Adam or SGD optimization algorithms, with a learning rate set from 0.001 to 0.01, a batch size set from 16 to 64, and a training epoch set from 30 to 100. Dropout is adopted to prevent overfitting, with a dropout rate set from 0.2 to 0.5.

In summary, the convolutional neural network adopts a ResNet-18 or ResNet-34 architecture with a depth of 18 and 34 layers, and effectively extracts spatial features through 3×3 or 5×5 convolution kernels. ReLU or Leaky ReLU activation functions combined with batch normalization accelerate training and enhance network stability. Residual connections effectively mitigate gradient vanishing, ensuring effective training of deep networks. The input layer receives feature images of 64×64 or 128×128 pixels, and rich spatial information is extracted through a plurality of convolutional and pooling layers. The training is performed by Adam or SGD optimizers with appropriate learning rates and batch sizes over 30 to 100 epochs. Dropout is adopted to prevent overfitting, improving model generalization and prediction accuracy.

Further, the long short-term memory network includes one to three LSTM layers, each layer includes 64 to 256 hidden units, and the input sequence length is set to 24 to 72 time steps, which corresponds to historical data of 24 to 72 hours. Information flow is controlled by a gating mechanism, including a forget gate, an input gate, and an output gate. The activation function is a tanh function. Gradient clipping is adopted to prevent gradient explosion, and the clipping threshold is set to 0.5 to 2.0. Network training is performed using a time backpropagation algorithm, the learning rate is set to 0.001 to 0.01, and a dropout ratio is set to 0.1 to 0.3 to prevent overfitting. An early stopping strategy is adopted, where training stops when the validation set loss does not decrease continuously for 5 to 10 epochs.

In summary, the long short-term memory network includes one to three LSTM layers, each layer includes 64 to 256 hidden units, and the input sequence covers historical data of 24 to 72 hours, thereby effectively capturing long-term dependencies in time series data. Information flow is adjusted by gating mechanisms, including a forget gate, an input gate, and an output gate, combined with a tanh activation function, thereby achieving precise modeling of temporal features. Gradient clipping is adopted to prevent gradient explosion, ensuring training stability. During training, optimization is performed using a time backpropagation algorithm, with an appropriate learning rate and dropout ratio set to prevent overfitting, and an early stopping strategy is adopted to effectively avoid excessive training, thereby improving model generalization capability and prediction accuracy.

Further, the attention mechanism adopts a multi-head self-attention structure, including four to eight attention heads, and each attention head has a dimension of 64. Attention weights are calculated through query, key, and value matrices by using a scaled dot-product attention mechanism, where the scaling factor is set to the square root of the dimension. Attention weights are normalized using a softmax function, and model performance is improved by using residual connections and layer normalization techniques. Positional encoding is generated using sine and cosine functions, and the maximum sequence length is set to 256. The multi-head attention mechanism captures dependencies between different time steps and different features, and an attention weight visualization system is established to intuitively display information of primary concern to the model.

In summary, the attention mechanism adopts a multi-head self-attention structure, including four to eight attention heads, and each head has a dimension of 64. Attention weights are calculated through query, key, and value matrices by using a scaled dot-product mechanism to enhance computational stability. The weights are normalized using a softmax function, and residual connections combined with layer normalization techniques are adopted, thereby effectively improving model training performance and generalization capability. Positional encoding is generated using sine and cosine functions, supporting a maximum sequence length of 256, thereby enhancing the capability of capturing dependencies between different time steps and different features within a sequence. Furthermore, the attention weight visualization system is established to intuitively display key information attended to by the model, thereby improving interpretability and tuning efficiency.

Further, the automatic adjustment of monitoring frequency and sampling strategy includes the following: Historical data related to the abnormality, including microbial activity fluctuations, abnormal changes of environmental parameters, and monitoring error records, are automatically retrieved according to the type of abnormality, and potential driving factors and impact scope of the abnormality are evaluated through a data fusion analysis module. For monitoring areas or monitoring points where abnormalities occur, the data acquisition frequency of relevant modules is adaptively increased according to the rate of environmental change and the intensity of the abnormality. When the spatial distribution of the abnormality exhibits an expanding trend, spatial weights and abnormality correlations of adjacent monitoring points are automatically identified, and temporary sampling points are added or backup sensor nodes are activated in necessary areas, thereby achieving spatially dense monitoring of abnormal regions. Sampling parameters are dynamically optimized according to the current environmental state and microbial response rate, including Raman laser power, electrochemical measurement frequency, and fluorescence exposure time, thereby improving data quality and reducing the influence of external interference on sampling stability.

Further, the evaluation of prediction accuracy adopts a diversified performance indicator system, including three dimensions: point prediction accuracy, interval prediction accuracy, and trend prediction accuracy. Point prediction accuracy is measured using root mean square error, mean absolute error, and mean absolute percentage error, where the root mean square error is required to be less than 10% of the actual value. Interval prediction accuracy is measured using prediction interval coverage and average interval width, where the coverage of a 95% confidence interval is required to be not less than 80%. Trend prediction accuracy is measured using directional accuracy and trend correlation coefficient, where the directional accuracy is required to be not less than 75%.

In summary, the evaluation of prediction accuracy adopts a multidimensional performance indicator system, thereby comprehensively measuring the capabilities of the model in point prediction, interval prediction, and trend prediction. The point prediction is evaluated using root mean square error, mean absolute error, and mean absolute percentage error, which ensures that the root mean square error is less than 10% of the actual value, thereby ensuring high numerical prediction accuracy. The interval prediction is measured by prediction interval coverage and average interval width, where the coverage of a 95% confidence interval is not less than 80%, thereby reflecting the reliability and confidence level of prediction results. The trend prediction is evaluated by directional accuracy and trend correlation coefficient, where the directional accuracy is not less than 75%, thereby ensuring accurate capture of change trends and effectively improving the practical value and decision-support capability of chlorite growth prediction.

It should be noted that user information (including, but not limited to, user device information and user personal information) and data (including, but not limited to, data used for analysis, stored data, and displayed data) involved in the present application are all information and data authorized by users or fully authorized by relevant parties. The collection, use, and processing of the relevant data comply with applicable regulations.

Those of ordinary skill in the art can understand that all or part of the processes in the methods of the above-described embodiments can be implemented by a computer program instructing relevant hardware. The computer program can be stored on a non-volatile computer-readable storage medium, and upon execution, can include processes according to the embodiments of the methods described above. In the embodiments provided in the present application, any reference to a memory, database, or other medium may include at least one of a non-volatile memory and a volatile memory. The non-volatile memory may include read-only memory (ROM), magnetic tape, floppy disk, flash memory, optical storage, high-density embedded non-volatile memory, resistive random-access memory (ReRAM), magnetoresistive random-access memory (MRAM), ferroelectric random-access memory (FRAM), phase change memory (PCM), graphene memory, or the like. The volatile memory may include random-access memory (RAM) or external high-speed cache memory. By way of illustration and not limitation, RAM may be in various forms, such as static random-access memory (SRAM) or dynamic random-access memory (DRAM).

In the embodiments provided in the present application, the databases involved may include at least one of relational databases or non-relational databases. The non-relational databases may include blockchain-based distributed databases or the like. This is not limited thereto. The processors involved in the embodiments provided in the present application may include a general-purpose processor, a central processing unit, a graphics processor, a digital signal processor, a programmable logic device, a data processing logic based on quantum computing, or the like. This is not limited thereto.

The technical features of the above embodiments may be combined in any manner. For the sake of conciseness, not all possible combinations of the technical features in the above embodiments are described. However, as long as the combinations of these technical features do not conflict, these technical features are considered to fall within the scope of the present application.

Specific examples are applied herein to illustrate the principles and embodiments of the present application. The description of the above embodiments is provided merely to facilitate understanding of the methods and core concepts of the present application. Those skilled in the art will recognize that changes may be made to specific embodiments and application ranges based on the principles of the present application. In summary, the contents of this specification should not be construed as limiting the scope of the present application.

What is claimed is:

1. A method for predicting chlorite growth based on microbial activity, comprising:
S1. generating a comprehensive data set by performing dynamic quantification monitoring of microbial activity and synchronous acquisition of environmental parameters based on a geological environment monitoring system, wherein the comprehensive data set comprises microbial activity indicators, environmental parameters, and chlorite growth data;
S2. establishing a kinetic model of microbial influence on chlorite nucleation and growth based on the comprehensive data set and molecular dynamics, and constructing a standardized training data set based on input data and output data of the kinetic model;
S3. constructing a spatiotemporal prediction model of microbial activity based on the standardized training data set; and
S4. dynamically updating and performing error correction on the spatiotemporal prediction model of microbial activity to obtain a final spatiotemporal prediction model, and outputting corrected chlorite growth prediction results based on the final spatiotemporal prediction model.

2. The method for predicting chlorite growth based on microbial activity according to claim 1, wherein generating the comprehensive data set by performing dynamic quantification monitoring of microbial activity and synchronous acquisition of environmental parameters based on the geological environment monitoring system in S1 is specifically as follows:
the geological environment monitoring system comprises a Raman spectroscopy detection module, an electrochemical impedance measurement module, a fluorescence probe labeling module, an environmental parameter monitoring module, and a data fusion analysis module; wherein
the Raman spectroscopy detection module is configured to identify characteristic peaks of microbial metabolites;
the electrochemical impedance measurement module is configured to detect changes in microbial film resistance;
the fluorescence probe labeling module is configured to label secretion states of extracellular polymers;
the environmental parameter monitoring module is configured to acquire environmental factor data; and
the data fusion analysis module is configured to establish a multi-source data fusion framework by integrating real-time monitoring data, historical geological data, and laboratory analysis results, and to construct a comprehensive data set comprising microbial activity indicators, environmental parameters, and chlorite growth data through the multi-source data fusion framework.

3. The method for predicting chlorite growth based on microbial activity according to claim 2, wherein the multi-source data fusion framework is specifically as follows:
the multi-source data fusion framework adopts a hierarchical fusion architecture, comprising sensor-level fusion, feature-level fusion, and decision-level fusion; wherein
the sensor-level fusion is configured to perform temporal registration, spatial registration, and data association processing on raw data from sensors of the same type;
the feature-level fusion is configured to extract correlated features and complementary information among different data sources, and to construct a multidimensional feature vector space; and
the decision-level fusion is configured to generate microbial activity evaluation results by combining fusion results from the sensor-level fusion and fusion results from the feature-level fusion.

4. The method for predicting chlorite growth based on microbial activity according to claim 1, wherein S2 specifically comprises:
predicting a complexation reaction mechanism between microbial extracellular polymers and chlorite precursor ions through molecular dynamics simulation, and verifying the prediction through chlorite synthesis experiments under laboratory-controlled conditions to establish a kinetic model of microbial activity on chlorite nucleation and growth,
wherein the kinetic model is configured to quantify an regulatory effect of microbial metabolites on chlorite crystal morphology and growth direction; and
constructing a standardized training data set based on the comprehensive data set, wherein the standardized training data set comprises temporal features of microbial activity, spatial distribution patterns, and environmental parameter variations, and
the standardized training data set is configured to capture the influence of environmental parameter changes on microbial community succession and a feedback regulatory mechanism of microbial activity fluctuations on chlorite growth patterns.

5. The method for predicting chlorite growth based on microbial activity according to claim 4, wherein constructing the standardized training data set specifically comprises:
organizing the comprehensive data set using a spatiotemporal gridding method, and dividing the comprehensive data set along a temporal dimension to obtain a plurality of time windows;
establishing a three-dimensional grid in a spatial dimension based on monitoring point locations, and adjusting the three-dimensional grid according to geological complexity of an monitoring area, wherein
each spatiotemporal grid unit comprises microbial activity indicators, environmental parameters, and chlorite growth data.

6. The method for predicting chlorite growth based on microbial activity according to claim 4, wherein the spatial distribution patterns specifically comprises:
spatial interpolation, spatial clustering, and spatial autocorrelation analysis; wherein
the spatial interpolation is configured to generate a continuous spatial distribution map based on microbial activity data from discrete sampling points;
the spatial clustering is configured to identify high-value zones, low-value zones, and transition zones of microbial activity and reveal the spatial distribution pattern of microbial activity; and
the spatial autocorrelation analysis is configured to quantitatively evaluate spatial correlation and spatial heterogeneity of microbial activity.

7. The method for predicting chlorite growth based on microbial activity according to claim 4, wherein establishing the kinetic model of microbial activity on chlorite nucleation and growth specifically comprises:
establishment of a nucleation kinetic model: calculating a nucleation rate equation comprising biological factors based on classical nucleation theory and non-classical nucleation theory by considering the influence of microbial metabolites on nucleation energy barrier, nucleation rate, and critical nucleus size;
establishment of a growth kinetic model: calculating growth rates of each crystal face of chlorite influenced by microbial extracellular polymers using helical growth mechanisms, two-dimensional nucleation mechanisms, and multi-step growth mechanisms; and
establishment of a morphology control model: predicting morphology evolution of chlorite crystals under microbial activity conditions based on Wulff configuration theory of crystal morphology by considering selective adsorption of organic molecules and surface energy modification effects.

8. The method for predicting chlorite growth based on microbial activity according to claim 1, wherein constructing the spatiotemporal prediction model of microbial activity based on the standardized training data set specifically comprises:
designing an adaptive deep learning architecture integrating a convolutional neural network, a long short-term memory network, and an attention mechanism; and
constructing the spatiotemporal prediction model of microbial activity by combining the standardized training data set with the adaptive deep learning architecture.

9. The method for predicting chlorite growth based on microbial activity according to claim 8, wherein the adaptive deep learning architecture specifically comprises:
a multi-layered structure comprising an input layer, a feature extraction layer, a feature fusion layer, a temporal modeling layer, and an output layer; wherein
the input layer is configured to standardize and encode the comprehensive data set and convert different types of data into vector forms processable by a network;
the feature extraction layer is configured to extract local features and global features by using a combination of one-dimensional convolutional neural networks and two-dimensional convolutional neural networks to process temporal data and spatial data, respectively;
the feature fusion layer is configured to adaptively fuse different types of local and global features using attention mechanisms and gating mechanisms, and generate fused features comprising microbial activity and environmental parameter information;
the temporal modeling layer is configured to model temporal dependencies of microbial activity and environmental parameters based on the fused features output from the feature fusion layer; and
the output layer is configured to output chlorite growth prediction results based on the temporal dependencies using fully connected layers and activation functions.

10. The method for predicting chlorite growth based on microbial activity according to claim 1, wherein dynamically updating and performing error correction on the spatiotemporal prediction model of microbial activity to obtain the final spatiotemporal prediction model specifically comprises:
generating the final spatiotemporal prediction model through dynamic updating and error correction of the spatiotemporal prediction model of microbial activity using Kalman filtering and particle filtering algorithms, comprising:
when prediction results indicate abnormal changes in chlorite growth rate or crystal morphology, triggering a warning mechanism of the geological environment monitoring system and adjusting monitoring frequency and sampling strategy;
when prediction results fall within a preset growth pattern range, maintaining a current monitoring strategy and continuously performing chlorite growth prediction; and
when prediction results exceed a preset range, increasing monitoring density and recalibrating prediction model parameters until prediction accuracy meets requirements.
